(19) United States
(12) Patent Application Publication
(10) Pub. No.: US 2010/0316529 A1
(43) Pub. Date: Dec. 16, 2010

(54) Anti-Microbial Systems and Methods

(76) Inventors: Peter Kite, Leeds (GB); David Hatton, Seattle, WA (US)

Correspondence Address: BUCHANAN, INGERSOLL & ROONEY PC, POST OFFICE BOX 1404, ALEXANDRIA, VA, 22313-1404, US

(21) Appl. No.: 12/820,007

(22) Filed: Jun. 21, 2010

Related U.S. Application Data
(63) Continuation of application No. 10/313,844, filed on Dec. 5, 2002.
(60) Provisional application No. 60/338,639, filed on Dec. 5, 2001.

(51) Int. Cl. A61L 2/18

(52) U.S. Cl. 422/28

(57) ABSTRACT

An antimicrobial system. When generally formulated to disinfect a catheter, the antimicrobial substance comprises water and between approximately 10 and 200 mg of tetrasodium EDTA for each milliliter of water. When formulated as a treatment solution for treating an infected catheter, the antimicrobial substance comprises water and between approximately 5 and 80 mg of tetrasodium EDTA for each milliliter of water. When formulated as a prophylactic substance for inhibiting infection of a catheter, the antimicrobial substance comprises water and between approximately 5 and 40 mg of tetrasodium EDTA for each milliliter of water.

### RELATED APPLICATIONS

This application is a continuation of U.S. application Ser. No. 10/313,844, which was filed on Dec. 5, 2002. U.S. application Ser. No. 10/313,844 claims priority of U.S. Provisional Application No. 60/338,639, which was filed Dec. 5, 2001. U.S. application Ser. No. 10/313,844 and U.S. Provisional Application No. 60/338,639 are herein expressly incorporated by reference.

### TECHNICAL FIELD

The present invention relates to anti-microbial systems and methods and, more specifically, to anti-microbial materials and delivery systems for applying anti-microbial materials.

### BACKGROUND OF THE INVENTION

In the following discussion, the terms “microbe” or “microbial” will be used to refer to microscopic organisms or matter, including fungal and bacterial organisms, and possibly including viral organisms, capable of infecting humans. The term “anti-microbial” will thus be used herein to refer to a material or agent that kills or otherwise inhibits the growth of fungal and/or bacterial and possibly viral organisms.

The term “disinfect” will be used to refer to the reduction, inhibition, or elimination of infectious microbes from a defined system. The term “disinfectant” will be used herein to refer to a one or more anti-microbial substances used either alone or in combination with other materials such as carriers, solvents, or the like.

The term “infected system” will be used herein to refer to a defined or discrete system or environment in which one or more infectious microbes are or are likely to be present. Examples of infected systems include a physical space such as a bathroom facility or operating room, a physical object such as food or surgical tool, a biological system such as the human body, or a combination of a physical object and a biological system such as a catheter or the like arranged at least partly within a human body. Tubes and other conduits for the delivery of fluids, in industrial and healthcare settings, may also define an infected system.

The need for anti-microbial agents in medical, dental, veterinarian, household, food preparation, industrial water supply, and other applications is well recognized. All of these environments may define infected systems in which infectious microbes may exist, such as on surfaces, in fluid conduits, and/or on humans or food for human consumption.

Conventionally, a number of anti-microbial systems and methods are used to disinfect infected systems. For example, disinfecting chemicals such as phenols and hyperchlorites are topically applied to infected surfaces; these disinfectant chemicals are often toxic to humans and thus must be handled, applied, and disposed of in a controlled manner. Other disinfecting systems and methods for non-biological physical objects include the application of heat and/or pressure such as in conventional autoclaving techniques.

### PRIOR ART

Ethylene diamine tetraacetic acid (EDTA) has been used for systemic detoxification treatment and as an anticoagulant in blood samples for some time. Thus its use for medical treatment and applications is established. The use of disodium EDTA and calcium disodium EDTA in combination with other compounds to enhance anti-microbial properties of these other compounds has been studied and practiced.

Most applications of EDTA are limited to the use of disodium EDTA or calcium EDTA. U.S. Pat. No. 5,688,516 to Raad et al. discloses the use of non-glycopeptide anti-microbial agents in connection with a second agent selected from the group of (a) an anticoagulant agent, (b) an anti-thrombogenic agent, and (c) a chelating agent. Possible chelating agents listed in the Raad patent include disodium EDTA and calcium disodium EDTA. Raad specifically states that EDTA may be excluded while still maintaining the therapeutic benefits of the disclosed invention and thus does not disclose the use of any form of EDTA by itself as an anti-microbial agent.

The Applicant's understanding of the references to EDTA in the literature are to disodium and calcium disodium salts of EDTA, with most of these references being to disodium EDTA. In addition, certain references in the literature discuss purported anti-microbial properties of disodium EDTA or calcium disodium EDTA when used in certain applications. Disodium EDTA has been used in low concentrations as an anticoagulant for blood samples; the concentrations of EDTA used in blood samples are too low to act as anything but an anti-coagulant.

### DETAILED DESCRIPTION OF THE INVENTION

The present invention is, in one form, an anti-microbial substance comprising certain salts of Ethylenediamine-tetraaceticacid (EDTA); these salts, which will be referred to herein as the disinfectant salts of EDTA, or more simply as the disinfectant salts, will be discussed initially below. In another form, the present invention comprises delivery systems and methods for applying the disinfectant salts of EDTA in specific environments. A number of such delivery systems and methods will be discussed in detail below.

### I. Disinfectant Salts of EDTA

In accordance with this invention it has been discovered that many stand-alone salts of Ethylenediamine-tetraaceticacid (EDTA) are effective anti-microbial agents and that specific salts are more effective than others. In particular, it has been discovered that other salts of EDTA exhibit anti-microbial (both antifungal and antibacterial) properties superior to those of the disodium salt in common use. In particular, dipotassium and ammonium EDTA are superior to disodium EDTA, and tetrasodium EDTA has been found to be preferred to disodium, ammonium, and dipotassium salts.

Infectious organisms often grow in biofilm systems that are commonly referred to as “slime”. Such biofilms have a mechanical structure in addition to a chemical or biochemical structure. The effects of these biofilms on disinfectant agents, systems, and methods have not been well understood. The Applicant believes that these biofilms function to protect at least some of the infectious organisms that form the biofilm. In particular, the biofilm can establish a protective “matrix” of glycocalyx, which induces a ‘biofilm resistance phenotype’, that protects the colonizing organisms within the biofilm by multiple up-regulation and down-regulation of genes.

The Applicant has discovered that the preferred disinfectant salts of EDTA are relatively effective in treating undesirable biofilms because they help to destroy the structure of the biofilm and allow the EDTA to kill or inhibit the growth of individual organisms within the biofilm.

The disinfectant salts of EDTA are commonly provided in crystalline powder form and in some cases in liquid form. The raw EDTA material may, in some situations, be used alone as a disinfectant, but is more likely to be used in an aqueous environment to create a disinfectant solution. While water is a typical solvent, other solvents may be used depending upon the specifics of the infected system. When used as a disinfectant solution, the disinfectant salts of EDTA may also be combined with other chemicals as dictated by the infected system. The exact form in which the EDTA is applied thus depends upon the specifics of the infected system.

The disodium and tetrasodium salts of EDTA are readily available, can be manufactured at reasonable cost, and are stable over time. These salts are generally considered to be non-toxic in small quantities and, when highly diluted, have been established as safe for human consumption and/or when used in contact with human blood, both in vitro and in vivo.

The dipotassium, ammonium, and other salts of EDTA are relatively expensive and less readily available than disodium and tetrasodium EDTA. In addition, the Applicant is not aware of any biocompatability information related to the disinfectant salts of EDTA other than disodium and tetrasodium EDTA. At present, then, from a commercial perspective, disodium and tetrasodium EDTA are generally preferred because of their availability, cost, stability, and known biocompatibility. However, dipotassium, ammonium, and other disinfectant salts of EDTA may be preferred in certain situations based on the details of the infected system or if manufacturing or biocompatability considerations should change.

The use of the disinfectant salts of EDTA as an anti-microbial agent thus shows significant promise in reducing the transmission of infectious microbes among humans.

### II. Application of Disinfectant Salts of EDTA to Exemplary Infected Systems

The disinfectant salts of EDTA may be used to disinfect a variety of types of infected systems. Each type of infected system will involve a delivery system to carry the EDTA molecule to the infectious microbe. The delivery system will often comprise one or more of a solvent that is combined with the disinfectant salt(s) to form a disinfectant solution and, typically, a physical structure for delivering the disinfectant solution to the infected system. Several examples of infected systems where use of the disinfectant salts of EDTA would be beneficial will be described below. It should be apparent however that the disinfectant salts of EDTA may have beneficial application to other types of infected systems, and the following discussion is not intended to limit the scope of the present application.

A. Conduits

It has been discovered that conduits can be treated with dipotassium, ammonium, or tetrasodium salts of EDTA as a preventative antiseptic or as treatment following potential fungal or bacterial infection.

Typically, the disinfectant salts of EDTA, when used to treat conduits, are dissolved in water. The following Table A sets forth typical concentrations, and ranges of these concentrations, of specified disinfectant salts of EDTA when used with water as a solvent. The concentrations represented in the following Table A are expressed in milligrams of EDTA per milliliter of water (mg/ml).

TABLE A

| | Typical | First | Second |
|---|---|---|---|
| | Preferred | Preferred | Preferred |
| | (approx.) | Range | Range |
| dipotassium | 100 mg/ml | 5-200 mg/ml | 5-1000 mg/ml |
| ammonium | 100 mg/ml | 5-200 mg/ml | 5-1000 mg/ml |
| tetrasodium | 100 mg/ml | 5-100 mg/ml | 5-1000 mg/ml |

The treatment of conduits can consist of locking, flushing, coating, or aerosol doses of the EDTA solution. Examples of conduits that may be treated using the disinfectant salts of EDTA include water lines in dental or medical offices, lines carrying sterile fluids, catheters or ports that carry blood and/or other fluids into and out of the body, industrial water supply lines which develop large biofilm populations which effect the efficient flow of fluids as well as contaminating the fluids passing through the line, and airway support devices. Other examples include consumption such as drink dispensers and food packaging. Conduits treated by the disinfectant salts of EDTA are typically made of plastic, but the principles of the present invention may be applied to conduit device made of any material such as metal that delivers or carries fluid.

B. Dissolving of Crystals in Medical Applications

A further discovery is the use of the disinfectant salts of EDTA in the treatment and prevention of crystal formation in or on urological catheters and in the treatment of renal stones in the bladder of renal patients by lock or by flushing with the described EDTA salt concentrations. The process dissolves the crystals of calcium and magnesium phosphates and also kills the bacteria producing urease which forms them. In particular, the disinfectant salts of EDTA have been found to kill the Proteus and Pseudomonas bacterial species, which are urease producers as well as other urinary pathogens.

Typically, the disinfectant salts of EDTA, when used to treat or prevent crystal formation in or on urological catheters, are dissolved in water. The following Table B sets forth typical concentrations, and ranges of these concentrations, of specified disinfectant salts of EDTA when used with water as a solvent in this application. The concentrations represented in the following Table B are expressed in milligrams of EDTA per milliliter of water (mg/ml).

TABLE B

| | Typical | First | Second |
|---|---|---|---|
| | treatment | Preferred | Preferred |
| | Preferred | Treatment | Inhibition |
| | (approx.) | Range | Range |
| dipotassium | 50 mg/ml | 30-100 mg/ml | 5-100 mg/ml |
| ammonium | 50 mg/ml | 30-100 mg/ml | 5-100 mg/ml |
| tetrasodium | 50 mg/ml | 30-100 mg/ml | 5-100 mg/ml |

C. Material Decontamination and Preservation

It has further been discovered that the stand-alone use of dipotassium, ammonium, or tetrasodium salts of EDTA decontaminates and preserves potentially infected materials such as blood and plasma and conduits and containers therefor. The disinfectant salts of EDTA may also be used at relatively high concentrations as a preservative for food and drink. Typically, the disinfectant salts of EDTA, when used as an additive for material decontamination or preservation, are dissolved in or applied to the surface of the material to be decontaminated and/or preserved.

The following Table C sets forth typical concentrations, and ranges of these concentrations, of specified disinfectant salts of EDTA when used to decontaminate and preserve blood. The concentrations represented in the following Table C are expressed in milligrams of EDTA per milliliter of blood (mg/ml).

TABLE C

| | Typical | First | Second |
|---|---|---|---|
| | Preferred | Preferred | Preferred |
| | (approx.) | Range | Range |
| dipotassium | 30 mg/ml | 10-50 mg/ml | 5-100 mg/ml |
| ammonium | 30 mg/ml | 10-50 mg/ml | 5-100 mg/ml |
| tetrasodium | 30 mg/ml | 10-50 mg/ml | 5-100 mg/ml |

The following Table D sets forth typical concentrations, and ranges of these concentrations, of specified disinfectant salts of EDTA when used to decontaminate and preserve liquids for human consumption. The concentrations represented in the following Table D are expressed in milligrams of EDTA per milliliter of the liquid to be treated (mg/ml).

TABLE D

| | Typical | First | Second |
|---|---|---|---|
| | Preferred | Preferred | Preferred |
| | (approx.) | Range | Range |
| dipotassium | 30 mg/ml | 5-100 mg/ml | 5-1000 mg/ml |
| ammonium | 30 mg/ml | 5-100 mg/ml | 5-1000 mg/ml |
| tetrasodium | 30 mg/ml | 5-100 mg/ml | 5-1000 mg/ml |

The following Table E sets forth typical concentrations, and ranges of these concentrations, of specified disinfectant salts of EDTA when used to decontaminate and preserve food for human consumption. In this application, the disinfectant salts of EDTA are typically dissolved in water to obtain a disinfectant solution that is sprayed on the food or in which the food is soaked. The concentrations represented in the following Table E are expressed in milligrams of EDTA per milliliter of water (mg/ml).

TABLE E

| | Typical | First | Second |
|---|---|---|---|
| | Preferred | Preferred | Preferred |
| | (approx.) | Range | Range |
| dipotassium | 30 mg/ml | 10-50 mg/ml | 5-1000 mg/ml |
| ammonium | 30 mg/ml | 10-50 mg/ml | 5-1000 mg/ml |
| tetrasodium | 30 mg/ml | 10-50 mg/ml | 5-1000 mg/ml |

D. Topical Applications to the Human Body

It has been discovered that the anti-microbial properties of dipotassium, ammonium, or tetrasodium salts of EDTA are effective in treatment of topical infections, including but not limited to skin, ear, anal, mouth, and vulvo/vaginal sites. One example is as an additive to a tooth paste.

The following Table F sets forth typical concentrations, and ranges of these concentrations, of specified disinfectant salts of EDTA when used as a topical disinfectant for humans. In this application, the disinfectant salts of EDTA are typically incorporated into a delivery system comprising a liquid diluent; such delivery systems may include creams, ointments, gels, and emulsions. The concentrations represented in the following Table F are expressed in milligrams of EDTA per milliliter of the diluent (mg/ml).

TABLE F

| | | First | Second |
|---|---|---|---|
| | Typical | Preferred | Preferred |
| | Preferred | Range | Range |
| dipotassium | 50 mg/ml | 50-100 mg/ml | 5-1000 mg/ml |
| ammonium | 50 mg/ml | 50-100 mg/ml | 5-1000 mg/ml |
| tetrasodium | 50 mg/ml | 50-100 mg/ml | 5-1000 mg/ml |

E. Topical Applications to Surfaces

The use of stand alone dipotassium, ammonium, or tetrasodium salts of EDTA has been discovered to be an effective disinfectant for surfaces and equipment in industrial, medical, and household applications. A typical infected system would include the walls, floors, and commode in a lavatory. The delivery system will typically comprise a solvent and tools that allow flushing, locking, wiping, soaking, fogging, or coating of the surface defining the infected system.

The following Table G sets forth typical concentrations, and ranges of these concentrations, of specified disinfectant salts of EDTA when used as a surface cleaner. In this application, the disinfectant salts of EDTA are typically incorporated into a delivery system comprising a liquid diluent or solvent, typically water. The concentrations represented in the following Table G are expressed in milligrams of EDTA per milliliter of the diluent or solvent (mg/ml).

TABLE G

| | | First | Second |
|---|---|---|---|
| | Typical | Preferred | Preferred |
| | Preferred | Range | Range |
| dipotassium | 100 mg/ml | 30-100 mg/ml | 10-1000 mg/ml |
| ammonium | 100 mg/ml | 30-100 mg/ml | 10-1000 mg/ml |
| tetrasodium | 100 mg/ml | 30-100 mg/ml | 10-1000 mg/ml |

F. Disinfection of Objects

The use of stand alone dipotassium, ammonium, or tetrasodium salts of EDTA in concentrations between one and 1000 mg/ml has been discovered to be an effective decontamination disinfectant for medical instruments and devices, dental (both consumer and professional) instruments and devices, and/or veterinary instruments and devices. A typical example would be a soak for disinfecting toothbrushes.

The following Table H sets forth typical concentrations, and ranges of these concentrations, of specified disinfectant salts of EDTA when used as a cleaner for objects. In this application, the disinfectant salts of EDTA are typically incorporated into a delivery system comprising a liquid diluent or solvent, typically water. The disinfectant solution is contained in a vessel, and the object to be disinfected is placed in the disinfectant solution for a given temperature and pressure, typically ambient, for a given exposure period. The length of the exposure period will depend upon the materials from which the object is made, the shape of the object, and the use of the object. The concentrations represented in the following Table H are expressed in milligrams of EDTA per milliliter of water (mg/ml).

TABLE H

| | | First | Second |
|---|---|---|---|
| | Typical | Preferred | Preferred |
| | Preferred | Range | Range |
| dipotassium | 100 mg/ml | 10-100 mg/ml | 10-1000 mg/ml |
| ammonium | 100 mg/ml | 10-100 mg/ml | 10-1000 mg/ml |
| tetrasodium | 100 mg/ml | 10-100 mg/ml | 10-1000 mg/ml |

G. Topical Disinfectant Solution for Contact Lenses

The use of stand alone disodium, dipotassium, ammonium, or tetrasodium salts of EDTA has been discovered to be an effective antiseptic solution for optical contact lenses.

The concentrations of the disinfectant salts of EDTA required to clean contact lenses in a reasonable period of time are typically high enough to cause eye irritation. Accordingly, the optical contact lens will typically also be exposed to a neutralizing agent after disinfection to reduce eye irritation. The neutralizing agent will typically be calcium chloride, but other neutralizing agents with similar properties may be used.

The following Table I sets forth typical concentrations, and ranges of these concentrations, of specified disinfectant salts of EDTA when used as a cleaner for contact lenses. In this application, the disinfectant salts of EDTA are typically incorporated into a delivery system comprising a liquid diluent or solvent, typically water. The disinfectant solution is contained in a vessel, and the contact lens to be disinfected is placed in the disinfectant solution for a given temperature and pressure, typically ambient, for a given exposure period. The concentrations represented in the following Table I are expressed in milligrams of EDTA per milliliter of water (mg/ml).

TABLE I

| | | First | Second |
|---|---|---|---|
| | Typical | Preferred | Preferred |
| | Preferred | Range | Range |
| dipotassium | 30 mg/ml | 5-100 mg/ml | 5-1000 mg/ml |
| ammonium | 30 mg/ml | 5-100 mg/ml | 5-1′000 mg/ml |
| tetrasodium | 30 mg/ml | 5-100 mg/ml | 5-1000 mg/ml |

H. Catheter Lock Details

The treatment of catheters with the disinfectant salts of EDTA falls under the general category of conduit treatment as described above but is of particular significance. Catheter devices include all conduits that are used to deliver fluids into or remove fluids from the human body. A subcutaneous port is considered a catheter for the purposes of the present invention.

The dipotassium, ammonium, or tetrasodium salts of EDTA has been discovered to be an effective treatment for catheters defining an infected system. The disinfectant salts of EDTA inhibit microbe colonization by treating the catheter with these salts at the prescribed concentration using a liquid lock prior to and in between infusions and/or by surface coating of catheter devices. A further application is the treatment of colonized or infected catheters by use of a liquid lock containing the disinfectant salts of EDTA in the preferred concentration and pH.

Typically, the disinfectant salts of EDTA, when used to treat catheters, are dissolved in water as a carrier, although other carriers may be used. Substances such as thrombolytics, sodium, alcohol, or reagents may also be added to the basic water/EDTA solution.

The following Tables J, K, and L set forth typical approximate concentrations, and ranges of these concentrations, of specified disinfectant salts of EDTA when used with water as a solvent or carrier. Table J is directed to the general disinfecting of cathethers. Table K is directed to the treatment of a catheter system that may be infected, while Table L is directed to a prophylactic solution designed to prevent infection. The concentrations represented in the following tables are expressed in milligrams of EDTA per milliliter of water (mg/ml).

TABLE J

| | | First | Second |
|---|---|---|---|
| | Typical | Preferred | Preferred |
| | Preferred | Range | Range |
| dipotassium | 100 mg/ml | 10-200 mg/ml | 5-1000 mg/ml |
| ammonium | 100 mg/ml | 10-200 mg/ml | 5-1000 mg/ml |
| tetrasodium | 100 mg/ml | 10-200 mg/ml | 5-1000 mg/ml |

TABLE K

| | | First | Second |
|---|---|---|---|
| | Preferred | Preferred | Preferred |
| | Treatment | Range | Range |
| tetrasodium | 40 mg/ml | 10-80 mg/ml | 5-100 mg/ml |

TABLE L

| | | First | Second |
|---|---|---|---|
| | Preferred | Preferred | Preferred |
| | Prophylactic | Range | Range |
| tetrasodium | 20 mg/ml | 10-80 mg/ml | 5-100 mg/ml |

### III. Clinical Tests

The Applicant has tested the efficacy of a number of disinfectant salts of EDTA against a number of microbes. The results of these tests are summarized in two tables attached hereto as Exhibit A1 and A2.

Exhibits A1 and A2 contain the results of clinical tests in which six different disinfectant salts of EDTA were each tested against a variety of microbes. Exhibit B contains a description of the test protocol used to obtain the conclusions set forth in the table of Exhibits A1 and A2.

The numbers contained in the Exhibit A2 table identify the minimum concentration of each disinfectant salt required to inhibit growth (MIC) of each of the tested microbes; the concentration is expressed as milligrams of disinfectant salt per milliliters of water (mg/ml).

The numbers contained in the Exhibit A1 table identify the minimum concentration of each disinfectant salt required to kill an entire population (MBC) of each of the tested microbes; the concentration is expressed as milligrams of disinfectant salt per milliliters of water (mg/ml).

Based on the test results as summarized in the Exhibit A1 and A2 tables, it can be seen that all of the tested disinfectant salts are effective to some degree against all of the listed microbes. However, based on a balance of factors including material costs, minimum concentration required for inhibitory and bactericidal effect over a broad spectrum of microbes, material availability, and the like, the Applicant concludes that tetrasodium EDTA demonstrates the most superior attributes.

Referring now to Exhibit C, contained therein is a table summarizing the results of clinical tests in which three different disinfectant salts of EDTA were each tested for bactericidal and inhibitory effect against a variety of yeasts. The protocol for agar dilution is described in Exhibit D appended hereto.

The bactericidal effects were measured as the Minimum Bactericidal Concentration (MBC), while the inhibitory effects were measured as the Minimum Inhibitory Concentration (MIC). In each case, the concentration was measured as milligrams of the disinfectant salt per milliliter of water (mg/ml).

Based on the test results as summarized in the Exhibit C table, it can be seen that the three tested disinfectant salts have both bactericidal and inhibitory effects against all of the listed yeasts. In particular, the Exhibit C table shows that the MIC values for Tetra-sodium are lower than the MIC values for the other two EDTA compounds. The liquifaction of the saborauds agar caused by the action of diammonium and dipotassium EDTA salts, prevented the transfer of the yeasts to the MBC plates, making it impossible to compare the MBC values using these agents. However, the Exhibit C table shows that the MBC values for tetra-sodium EDTA are comparable to the MBC values for the Gram negative and Gram positive organisms.

From the foregoing, it should be clear that the present invention may be embodied in forms other than those discussed above; the scope of the present invention should be determined by the following claims and not the detailed discussion presented above.

### EXHIBIT A1

EXHIBIT A1

| | Cupric | | | | Magnesium | |
|---|---|---|---|---|---|---|
| | disodium | Dipotassium | Diammonium | Tetrasodium | disodium | Ferric-sodium |
| Organism | EDTA | EDTA | EDTA | EDTA | EDTA | salt EDTA |
| S. epidermidis | <0.5 | 8 | 4 | 1 | 30 | >30 |
| S. epidermidis | <0.5 | 8 | 8 | 2 | 30 | >30 |
| S. xylosus | <0.5 | 6 | 4 | 25 | >30 | >30 |
| S. capitis | <0.5 | 10 | 8 | 6 | >30 | >30 |
| S. lentus | <0.5 | 10 | 10 | 30 | >30 | >30 |
| S. capitis | <0.5 | 8 | 10 | >30 | >30 | >30 |
| S. simulans | <0.5 | 8 | 10 | 30 | >30 | >30 |
| S. aureus | <0.5 | 6 | 6 | 30 | >30 | >30 |
| S. aureus | <0.5 | 8 | 10 | >30 | >30 | >30 |
| S. aureus | <0.5 | 6 | 15 | >30 | >30 | >30 |
| S. aureus | <0.5 | 8 | 15 | <0.5 | >30 | >30 |
| S. aureus | <0.5 | 8 | 10 | 8 | >30 | >30 |
| MRSA | <0.5 | 6 | 8 | 8 | >30 | >30 |
| MRSA | <0.5 | 10 | 6 | 10 | >30 | >30 |
| MRSA | <0.5 | 8 | >15 | >30 | >30 | >30 |
| MRSA | <0.5 | 8 | 10 | 30 | >30 | >30 |
| MRSA | <0.5 | 8 | 10 | 25 | >30 | >30 |
| VRE | <0.5 | 8 | 15 | 15 | >30 | >30 |
| VRE | >30 | 8 | >15 | >30 | >30 | >30 |
| Ent. faeculm | <0.5 | 8 | 15 | >30 | >30 | >30 |
| Ent. faecalis | <0.5 | 15 | 15 | 10 | >30 | >30 |
| Kleb. pnemoniae | >30 | 15 | >10 | 15 | >30 | >15 |
| Kleb. pnemoniae | >30 | 15 | >10 | 15 | >30 | >15 |
| Kleb. oxytoca | >30 | 15 | >10 | 8 | >30 | >15 |
| Kleb. ornitholytica | >30 | >15 | >10 | 30 | >30 | >15 |
| E. coli | >30 | >15 | >10 | 10 | >30 | >15 |
| E. coli | >30 | 15 | >10 | 1.5 | >30 | >15 |
| E. coli | >30 | 15 | >10 | 10 | >30 | >15 |
| Ent. cloacae | >30 | 15 | >10 | 20 | >30 | >15 |
| Ent. cloacae | >30 | >15 | >10 | 20 | >30 | >15 |
| Ent. cloacae | 8 | >15 | >10 | 20 | >30 | >15 |
| Steno. maltophilia | >30 | 10 | >10 | 4 | >30 | >15 |
| Pseudomonas | >30 | >15 | >10 | 20 | >30 | >15 |
| aeruginosa | | | | | | |
| Pseudomonas | >30 | >15 | >10 | 20 | >30 | >15 |
| aeruginosa | | | | | | |
| Pseudomonas sp. | >30 | 15 | >10 | 30 | >30 | >15 |
| Cornyeform | <0.5 | <0.5 | 1 | <0.5 | 4 | 10 |
| amycolatum | | | | | | |
| Coryneform | <0.5 | <0.5 | 1 | <0.5 | 4 | 10 |
| strait/amy | | | | | | |
| Acinetobacter | <0.5 | <0.5 | <0.5 | <0.5 | <0.5 | 10 |
| baumanii | | | | | | |
| Acinetobacter | 15 | >15 | >10 | 2 | >30 | >15 |
| baumanii | | | | | | |
| Proteus mirabilis | >30 | >15 | >10 | 20 | >30 | >15 |
| Proteus vulgaris | >30 | >15 | >10 | 20 | .30 | >15 |
| Proteus mirabilis | >30 | 15 | >10 | 20 | >30 | >15 |

### EXHIBIT A2

EXHIBIT A2

| | Cupric | | | | Magnesium | |
|---|---|---|---|---|---|---|
| | disodium | Dipotassium | Diammonium | Tetrasodium | disodium | Ferric-sodium |
| Organism | EDTA | EDTA | EDTA | EDTA | EDTA | salt EDTA |
| S. epidemridis | <0.5 | <0.5 | <0.5 | 1 | 6 | >20 |
| S. epidermidis | <0.5 | <0.5 | <0.5 | 1 | 6 | >20 |
| S. xylosus | <0.5 | <0.5 | <0.5 | <0.5 | 2 | >20 |
| S. capitis | <0.5 | <0.5 | <0.5 | <0.5 | 1.5 | >20 |
| S. lentus | <0.5 | <0.5 | <0.5 | 1 | 6 | >20 |
| S. capitis | <0.5 | <0.5 | <0.5 | 1 | 6 | >20 |
| S. simulans | <0.5 | <0.5 | <0.5 | 1 | 1.5 | >20 |
| S. aureus | <0.5 | 1 | 1 | <0.5 | >30 | >20 |
| S. aureus | <0.5 | 1 | 1 | 1 | >30 | >20 |
| S. aureus | <0.5 | 1 | 1 | 1 | >30 | >20 |
| S. aureus | <0.5 | 1 | 1 | 1 | >30 | >20 |
| S. aureus | <0.5 | 1 | 1 | <0.5 | >30 | >20 |
| MRSA | <0.5 | 1 | 1 | 1 | >30 | >20 |
| MRSA | <0.5 | 1 | 1 | 1 | >30 | >20 |
| MRSA | <0.5 | 1 | 1 | 1 | >30 | >20 |
| MRSA | <0.5 | 1 | 1 | 1 | >30 | >20 |
| MRSA | <0.5 | 1 | 1 | 1 | >30 | >20 |
| VRE | <0.5 | <0.5 | <0.5 | 1 | 25 | 2 |
| VRE | >30 | 1 | 1 | 1 | >30 | >20 |
| Ent. faecuim | <0.5 | <0.5 | <0.5 | <0.5 | 1.5 | 4 |
| Ent. faecalis | <0.5 | 1 | 1 | 1 | >30 | 4 |
| Kleb. pnemoniae | >30 | 1.5 | 4 | 8 | >30 | >15 |
| Kleb. pnemoniae | >30 | 1 | 1.5 | 4 | >30 | 15 |
| Kleb. oxytoca | >30 | 1 | 1 | 4 | >30 | >15 |
| Kleb. ornitholytica | >30 | 1 | 1 | 4 | >30 | 15 |
| E. coli | >30 | 1 | 1 | 4 | >30 | 15 |
| E. coli | 6 | 1 | 1.5 | 1 | >30 | >15 |
| E. coli | >30 | 1 | 4 | 4 | >30 | >15 |
| Ent. cloacae | >30 | 4 | 4 | 8 | >30 | >15 |
| Ent. cloacae | >30 | 4 | 4 | 10 | >30 | >15 |
| Ent. cloacae | 6 | 6 | <0.5 | 8 | >30 | >15 |
| Steno. maltophilia | 6 | <0.5 | 1 | 1 | >30 | 10 |
| Pseudomonas | >30 | 1 | 1 | 2 | >30 | 15 |
| aeruginosa | | | | | | |
| Pseudomonas | >30 | 1 | 1 | 2 | >30 | 15 |
| aeruginosa | | | | | | |
| Pseudomonas sp. | >30 | 1 | <0.5 | 2 | >30 | 15 |
| Cornyeform | O.5 | <0.5 | <0.5 | <0.5 | <0.5 | 10 |
| amycolatum | | | | | | |
| Coryneform | <0.5 | <0.5 | <0.5 | <0.5 | <0.5 | 10 |
| strait/amy | | | | | | |
| Acinetobacter | <0.5 | <0.5 | <0.5 | <0.5 | <0.5 | 6 |
| baumanii | | | | | | |
| Acinetobacter | 6 | <0.5 | <0.5 | 1 | >30 | >15 |
| baumanii | | | | | | |
| Proteus mirabilis | 6 | <0.5 | <0.5 | 1 | >30 | >15 |
| Proteus vulgaris | >30 | 1 | <0.5 | 4 | >30 | >15 |
| Proteus mirabilis | 6 | <0.5 | <0.5 | 1 | >30 | >15 |

### EXHIBIT B

### Exhibit B

### Protocol for EDTA Evaluations

Protocol one: Broth dilutions:

Inoculate a 25 ml nutrient broth (Oxoid No2) in a sterile universal container with one of the following organisms; Pseudomonas aeruginosa, Proteus Mirabilis, E. coli, Klebsiella pneumoniae, Staph aureus, Coagulase negative staphylococci, Enterococcus faecalis or MRSA.

Incubate the broth at 37° C. overnight.

Next day make up a series of nutrient broths containing a dilution of the appropriate EDTA as in the following list; 0 mg/mL, 0.25 mg/mL, 0.5 mg/mL, 1.0 mg/mL, 1.5 mg/mL, 2.0 mg/mL, 4.0 mg/mL, 6.0 mg/mL, 8.0 mg/mL, 10 mg/mL, 15 mg/mL, 20 mg/mL, 25 mg/mL, 30 mg/ml, 40 mg/mL, 50 mg/mL, 60 mg/mL, 70 mg/mL, 80 mg/mL, 90 mg/mL, 100 mg/mL.

To make these concentrations put the EDTA needed for 10 mLs in only 9 mLs of broth in sterile universal containers, as this will be diluted further when the organisms are added.

Dilute the overnight broth cultures to 10⁷ organisms/mL using more sterile nutrient broth.

Add 1 mL of this diluent to each of the EDTA series broths. This gives a final concentration of 10⁶ organism/mL in the EDTA broths.

Incubate for 18 hours at 37° C.

Next day visually read the MIC's by observing the turbidity of the broths.

Plate out 1 uL of each dilution onto fresh blood agar plate and spread using a sterile plastic spreader.

Incubate overnight at 37° C.

Next day read the MBC's by performing colony counts.

### EXHIBIT C

EXHIBIT C

| | Di-ammonium | Di-ammonium | Di-potassium | Di-potassium | Tetra-sodium | Tetra-sodium |
|---|---|---|---|---|---|---|
| Organism | EDTA (MIC's) | EDTA (MBC's)* | EDTA (MIC's) | EDTA (MBC's)* | EDTA (MIC's) | EDTA (MBC's) |
| C. albicans | 0.5 | ND | 0.5 | ND | 0.5 | 15 |
| C. albicans | 0.5 | ND | 0.5 | ND | 0.5 | 15 |
| C. albicans | 0.5 | ND | 0.5 | ND | 0.5 | 0.5 |
| C. lucitaniae | 0.5 | ND | 0.5 | ND | 0.5 | 6 |
| C. tropicalis | 1 | ND | 1 | ND | 0.5 | 10 |
| C. guilliermondii | 0.5 | ND | 0.5 | ND | 0.5 | 0.5 |
| C. glabrata | 0.5 | ND | 0.5 | ND | 0.5 | 2 |
| C. parapsilosis | 0.5 | ND | 0.5 | ND | 0.5 | 8 |
| C. glabrata | 0.5 | ND | 0.5 | ND | 0.5 | 8 |

*Unable to perform >0.1 mg/ml as agar was dissolved by agent

### EXHIBIT D

### Exhibit D

### EDTA Agar Dilution Method

Protocol 3: Agar dilutions.

Method:

Prepare nutrient agar plates with increasing concentrations of EDTA, 0, 0.5, 1, 1.5, 2, 4, 6, 8, 10, 15, 20, 25, 30 mg/mL, using the following method.
Pour 300 mL distilled water into a sterile glass bottle.
Add the appropriate amount of EDTA. This is the concentration desired ×300, in grams.
Once the EDTA has fully dissolved, add 8.4 g of Nutrient agar powder (Oxoid). Mix thoroughly.
Do this for each concentration of EDTA required. Autoclave all the media at 121° C. for 20 mins.
To pour the plates the media is remelted using a hot water bath at 100 C.
Pour 20 mL agar into a sterile petri dish and allow to set. Do this until all the media is used up. Label the plates with the concentration of EDTA they contain.
These plates can then be stored, until they are needed, in a 4° C. fridge.
Use an automatic plate inoculator to inoculate each plate with 21 organisms. The template for these inoculations is show in the attached diagrams.
Incubate the plates overnight at 37° C.
Next day score + or − for growth.
Use the automatic plate inoculator to transfer the growth from the initial plates to replica plates to determine the MBC's.
Incubate the replica plates overnight at 37° C.
Next day score + or − for growth.

1. A method for disinfecting a catheter by contacting the catheter with a disinfectant solution consisting essentially of ethylene diamine tetraacetic acid (EDTA) salt and a solvent, wherein the EDTA salt is at a concentration from about 10 to about 80 mg for each ml of solvent, and wherein the EDTA salt comprises tetrasodium EDTA.

2. The method of claim 1, wherein the solvent is water.

3. The method of claim 1, wherein contacting the catheter with the disinfectant solution is accomplished by locking, flushing or coating the catheter with the disinfectant solution.

4. The method of claim 1, wherein the conduit is a urological catheter.

5. A method for disinfecting a catheter comprising:
introducing a disinfectant solution into an interior lumen of the catheter, wherein the disinfectant solution consists essentially of EDTA salt and a solvent, wherein the EDTA salt is at a concentration from about 10 to about 80 mg for each ml of solvent, and wherein the EDTA salt comprises tetrasodium EDTA;
holding the disinfectant solution within the lumen for a selected period of time; and
removing the disinfectant solution from the interior lumen.

6. A method for disinfecting a catheter comprising:
introducing a disinfectant solution into an interior lumen of the catheter, wherein the disinfectant solution consists of EDTA salt and a solvent, wherein the EDTA salt is at a concentration from about 5 to about 40 mg for each ml of solvent, and wherein the EDTA salt comprises tetrasodium EDTA;
holding the disinfectant solution within the lumen for a selected period of time; and
removing the disinfectant solution from the interior lumen.
